(19) United States
(12) Patent Application Publication
(10) Pub. No.: US 2004/0256581 A1
(43) Pub. Date: Dec. 23, 2004

(54) Hand-held ultraviolet sterilization lamp

(76) Inventors: David Au, Kowloon (HK); Wayne Tang, Irving, CA (US)

Correspondence Address: BOHAN, MATHERS & ASSOCIATES, LLC, PO BOX 17707, PORTLAND, ME, 04112-8707, US

(21) Appl. No.: 10/600,405

(22) Filed: Jun. 20, 2003

(51) Int. Cl. A61L 2/10

(52) U.S. Cl. 250/504 H

(57) ABSTRACT

A handheld, battery-operated ultraviolet sterilization device having a cold cathode ultraviolet lamp enclosed in a protective chassis. The cold cathode ultraviolet lamp is a low voltage lamp and the power supplied by two AA batteries provides hours of lamp operation.

### BACKGROUND INFORMATION

1. Field of the Invention

The invention relates to the field of sterilizing surfaces with ultraviolet light. More particularly, the invention relates to hand-held battery-operated ultraviolet sterilization devices.

2. Description of the Prior Art

Bacteria and viruses are susceptible to an ultraviolet wavelength of 254 nm and it is known to use ultraviolet light to destroy harmful pathogens. Ultraviolet light has long been used in hospital and laboratory environments that are traditionally very concerned about maintaining sterile conditions. The equipment used for ultraviolet sterilization in such settings is geared to sterilizing large amounts of articles or large surface areas. Accordingly, such equipment is stationary and generally requires a large amount of power and/or is very expensive.

It is not only the hospital and laboratory environments that are interested in controlling exposure to pathogens. In many situations, a person may want to sterilize a surface in a public area, such as a toilet seat, eating surfaces, public telephone surfaces, etc., particularly in areas where there is an outbreak of a highly contagious disease. Thus, it is desirable to have a hand-held, lightweight sterilization device that is effective in destroying pathogens, yet is safe and easy to use.

Conventional ultraviolet lamps in portable devices are either quartz or fluorescent lamps. These lamps consume a large amount of power. For example, the smallest fluorescent lamp capable of producing 254 nm ultraviolet light that is currently available is a lamp provided by TECWEST U.S.A., Inc. The lamp is 134.5 mm long and 15.5 mm in diameter and draws 0.162 A current at 120 Volts. The input power is, thus, 8.5 Watts. The average rating of a conventional AA battery is 2,000 milliamp-hours. Using 2 AA batteries in series, the power required to operate such a lamp would drain the batteries in less than 1 hour. Handheld devices using such lamps either require large batteries or require battery replacement after only a brief operating time. Furthermore, quartz or fluorescent ultraviolet lamps run hot due to the large amount of power they consume. This presents a safety issue, because a user runs the risk of getting burned when using the lamp.

Hand-held ultraviolet sterilization devices are known. U.S. Pat. No. 4,952,369 (Belilos; 1990) discloses an ultraviolet flashlight comprising an ultraviolet light source, a power source, an on/off switch and a gravity switch. Turning the on/off switch to on activates the flashlight. The gravity switch breaks the circuit to the light source if the flashlight is not facing downward. The beam of the ultraviolet light source is directed over a surface to be sterilized. This device requires a battery pack that provides 3.0 Volts DC and uses a fluorescent or quartz type ultraviolet lamp. As described above, the lamp requires a high amount of power for a battery-operated device and, thus, the number of operating hours that the 3 volt battery pack provides is very limited.

Exposure to ultraviolet light is harmful to humans and most sterilization devices are equipped with safety features to limit this exposure. One safety feature provides a protective shield that surrounds the ultraviolet light and protects users from exposure. Another safety feature prevents the device from being turned on inadvertently. U.S. Pat. No. 5,920,075 (Whitehead; 1999) discloses an ultraviolet sterilization device that has an electronic safety mechanism that includes both a switch lock circuit and a digital lock circuit that must be actuated to close an electric circuit and actuate the device. U.S. Patent Application Publication US 2001/0042842 (Leighley et al.; 2001) discloses a hand-held germicidal lamp with a safety switch that utilizes an activation tool. Additionally, Leighley et al. discloses a second safety measure in the form of a switch cover that must be pushed aside in order to get to the safety switch. Both the Whitehead and Leighley et al. devices require some type of key for actuation. Using a key is time consuming and presents a disadvantage for someone who would like to quickly use the device to scan a dining table in a restaurant. Another disadvantage of using a key is that, if lost or unavailable, the device is inoperable.

What is needed, therefore, is a hand-held, lightweight, battery-operated sterilization device that uses an energy-efficient ultraviolet light source. What is further needed is such a device that is safe for a user to operate without risking being burned. What is yet further needed is such a device that includes a safety mechanism that prevents use by children, but is quickly activatable by adults.

### BRIEF SUMMARY OF THE INVENTION

For the reasons cited above, it is an object of the present invention to provide a hand-held, lightweight battery-operated sterilization device that uses an energy-efficient ultraviolet lamp. It is a further object to provide such a device that remains cool to the touch when operated. It is a yet further object to provide such a device that prevents use by children, but is quickly actuatable by adults.

The objects of the invention are achieved by providing an ultraviolet sterilization device comprising a cold cathode gas discharge ultraviolet lamp and a battery source. A suitable lamp is a commercially available cold cathode ultraviolet lamp, such as is distributed by Gilway Technical Lamp. The use of cold cathode lamps is known in other industries. Such lamps are available as visible-light and ultraviolet-light lamps. Cold cathode ultraviolet lamps are available with a peak wavelength of either 254 nm or 350 nm and consume only a fraction of the power of conventional quartz or fluorescent ultraviolet lamps. A tubular cold cathode ultraviolet lamp emits a short-wave ultraviolet light at 254 nm and has a typical operating voltage of between 100 and 200 volts. A suitable lamp, for example, is the FL9030-UV254 lamp provided by Gilway Technical Lamp, having a length of 30.0 mm and a diameter of 3.0 mm. The lamp draws 5 milliamps at 160 volts. Two AA batteries provide sufficient energy to power the lamp for 7.5 hours. With the ultraviolet sterilization device according to the invention, a small 3 volt battery-operated inverter provides input power for the ultraviolet lamp. The power consumption is extremely low. For example, the inverter produces the necessary operating voltage of between 100 and 200 volts at approximately 5 milliamperes of current. Because of the low power consumed by the cold cathode ultraviolet lamp, the lamp does not get hot. This minimizes the risk that a user of the device will experience discomfort or risk burning when operating the lamp. Because of the energy efficiency, other alternative power sources, such as solar-powered batteries, or lithium batteries, are also suitable as energy sources for the ultraviolet device according to the invention.

The ultraviolet sterilization device according to the invention has a chassis, which encloses the cold cathode ultraviolet lamp, the inverter, and the battery pack. The chassis has a window to allow the ultraviolet light to project outward from device, and may be provided with a retractable shield to protect the lamp from damage by impact or scratching. Preferably, the chassis is made of aluminum or other suitable material. If made of a material that is not reflective, a reflector lines the inner wall of the chassis behind the lamp, as means of increasing increasing the amount of ultraviolet light that is projected outward through the window. If made of aluminum, or other reflective material, the area behind the lamp may be polished to increase its reflective properties. Access to the battery pack is provided by any number of conventional means, such as a threaded plug, a sliding cover, etc. Mounted on the chassis is an on/off switch that completes or interrupts the electrical circuit between the power source and the lamp.

Means for preventing a child from using the ultraviolet sterilization device are important if the device is to be generally available for use in public areas. Many child safety features that are used on various other products, such as for pill bottles and cigarette lighters, are also suitable as safety features for the ultraviolet sterilization device according to the invention. Such features usually require a certain dexterity that is generally not achievable by a young child. One such child-safety feature is disclosed in U.S. Pat. No. 6,077,070 (Doucet et al; 2000), which teaches a safety mechanism that requires a physical displacement in two directions in order to light a cigarette lighter. This particular mechanism cannot remain displaced without continuous application of force by the user. Once force is removed then the device returns to a locked state. The ultraviolet sterilization device according to the invention may also be equipped with a gravity switch, such that the circuit to the lamp is interrupted if the light is not exposed downward. If desired, the ultraviolet sterilization device may also be equipped with a safety switch that requires the use of a key.

The particular shape of the ultraviolet sterilization device according to the invention is not critical to the invention. Because the device is so small and lightweight, a pen-like shape is quite suitable, as it is easily carried and fits into many small carrying cases, such as a handbag or a jacket pocket. Another suitable shape is one that is flat and oblong and that fits easily in the palm of the hand of the user. Whichever shape is used for the chassis, it is preferably one that is conducive to holding the ultraviolet sterilization device in one hand, with the ultraviolet light discharge window facing downward. By depressing the on/off switch and sweeping the device across the surface to be disinfected for a brief period of time, e.g., for five to ten seconds, the user is easily and quickly able to disinfect seating or eating surfaces, food preparation surfaces, food items, flatware, containers, and other common items encountered in public or in the home that are suspected of containing harmful pathogens.

### BRIEF DESCRIPTION OF THE DRAWINGS

FIG. 1 is a perspective view of the first embodiment of the ultraviolet sterilization device according to the invention.

FIGS. 2A and 2B illustrate a child safety feature.

FIGS. 3A and 3B illustrate the ultraviolet sterilization device according to the invention with a different shape chassis.

### DETAILED DESCRIPTION OF THE INVENTION

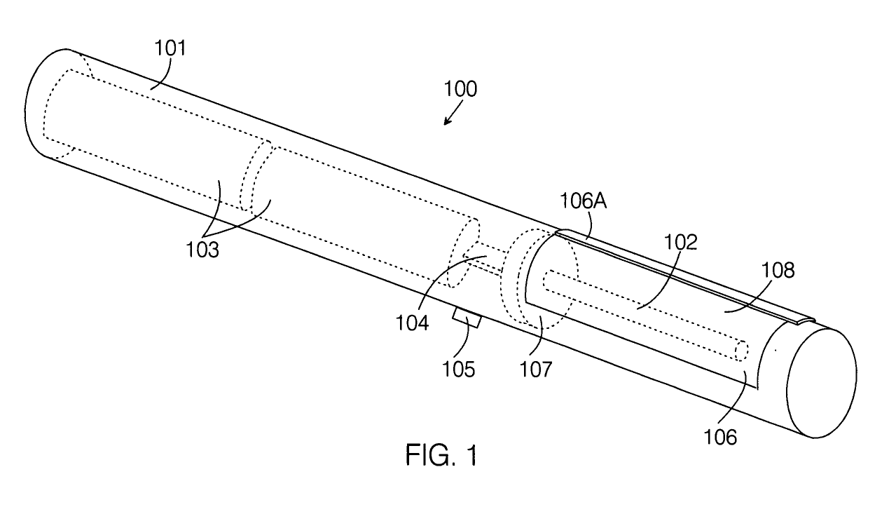

FIG. 1 illustrates the ultraviolet sterilization device 100 according to the invention. A protective chassis 101 houses a cold cathode ultraviolet lamp 102 and a battery pack 103, which preferably comprises two M batteries. A small battery-operated inverter 104 converts the low DC voltage from the power supply 103 to the higher AC voltage required to power the cold cathode ultraviolet lamp 102. The chassis 101 is provided with a window 106 through which the light from the cold cathode ultraviolet lamp 102 is projected outward to the surface to be disinfected. The window 106 may be protected by a protective shield 106A that is retractable when the ultraviolet sterilization device 100 is ready for operation. A reflector 108 is provided in the chassis 101 behind the lamp 102. In the embodiment shown, the chassis 101 is made of aluminum and the reflector 108 is a polished inner surface area of the chassis 101.

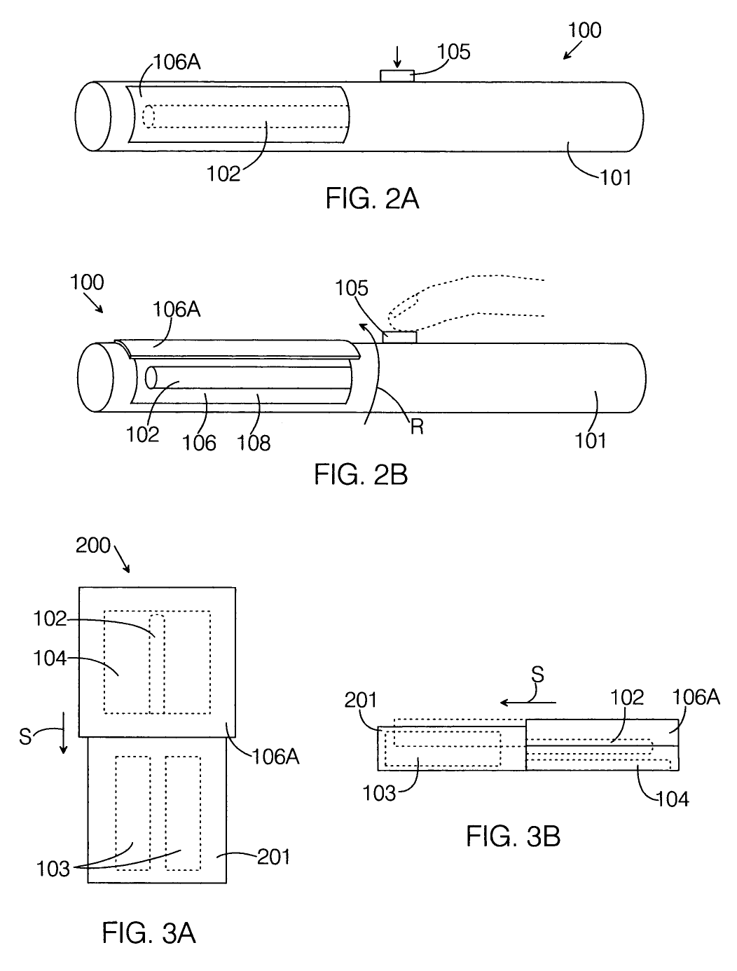

FIGS. 2A and 2B illustrate an example of a child-safety feature on the ultraviolet sterilization device 100. FIG. 2A shows the protective shield 106A in a closed position. Continuously depressing the on/off switch 105 while turning the protective shield 106A in a clockwise direction, illustrated by directional arrow R closes the electric circuit and actuates the lamp 102. The window 106 is now exposed and light from the cold cathode ultraviolet lamp 102 is projected outward, as illustrated in FIG. 2B.

FIGS. 3A and 3B illustrate a ultraviolet sterilization device 200 that is the same as the embodiment of the device 100 according to the invention, but with a chassis 201 of a different shape. All the components of the device 200 are identical to those discussed above with the device 100. The protective shield 106A is slidable back over a portion of the chassis 201 as indicated by the directional arrow S to expose the window 106.

What is claimed is:

1. A handheld ultraviolet sterilization device comprising:
a cold cathode ultraviolet lamp;
a chassis having a light exposure window;
a power inverter; and
a DC power source;
wherein said lamp, said power inverter, and said DC power source are electrically connected to each other and are encased within said chassis.

2. The device of claim 1, wherein said light exposure window is protected by a retractable protective shield.

3. The device of claim 1, wherein said power source is an M battery source.

4. The device of claim 3, wherein said AA battery source includes two AA batteries.

5. The device of claim 1, wherein said power source is a lithium battery.

6. The device of claim 1, wherein said power source is

7. The device of claim 1 further comprising a child-safety features.

8. The device of claim 8 further comprising a first safety component and a second safety component, wherein said child-safety feature is a keyless feature that requires simultaneous physical displacement of first safety component and said second safety component for said ultraviolet lamp to be operable.

9. The device of claim 8, wherein said first safety component is an on/off switch and said second safety component is an electrical connection to said protective shield, and wherein said child-safety feature is operable only when said on/off switch is depressed while said protective shield is moved to an open position.

10. The device of claim 1 further comprising a reflector arranged within said chassis behind said lamp, so as to reflect light from said lamp outward through said light exposure window.
